(19) United States
(12) Patent Application Publication
(10) Pub. No.: US 2022/0248776 A1
(43) Pub. Date: Aug. 11, 2022

(54) BIOSENSING TEXTILE AND METHOD OF MAKING THE SAME

(71) Applicant: Prevayl Innovations Limited, Manchester (GB)

(72) Inventor: Isabel Rose Hepworth, Manchester (GB)

(21) Appl. No.: 17/614,255

(22) PCT Filed: Jul. 10, 2020
(86) PCT No.: PCT/GB2020/051663

§ 371 Date: Nov. 24, 2021

(30) Foreign Application Priority Data
Jul. 12, 2019 (GB) 1910049.4
Apr. 14, 2020 (GB) 2005398.9

(51) Int. Cl. A41D 1/00; A41D 13/12; A61B 5/00

(52) U.S. Cl. CPC A41D 1/005; A61B 5/6804; A41D 13/1281

(57) ABSTRACT

The method comprises providing a textile patch (103) comprising a biosensing unit (101a, b). The method comprises providing a controller (105) for controlling the biosensing unit (101a,b) ono a surface of the textile patch (103). The method comprises attaching the textile patch (103) to a textile panel (107) to form the biosensing textile. The controller (105) is sandwiched between the textile patch (103) and the textile panel (107). The textile panel (107) may be attached to the inside of a garment (200). A garment and textile panel are also provided.

### CROSS REFERENCE TO RELATED APPLICATIONS

This application claims priority from United Kingdom Patent Application number 1910049.4 filed on 12 Jul. 2019 and United Kingdom Patent Application number 2005398.9 filed on 14 Apr. 2020, the whole contents of which are incorporated herein by reference.

### BACKGROUND

The present invention is directed towards a biosensing textile, a biosensing garment comprising the biosensing textile and a method of making the same.

Garments incorporating sensors are wearable electronics designed to interface with a wearer of the garment, and to determine information such as the wearer's heart rate, rate of respiration, activity level, and body positioning. Such properties can be measured with a sensor assembly that includes a sensor for signal transduction and/or microprocessors for analysis. Such garments are commonly referred to as ‘biosensing garments’ or ‘smart clothing’.

It is desirable to provide an improved process for incorporating electronic components into the biosensing garment.

### SUMMARY

According to the present disclosure there is provided a method of manufacturing a biosensing textile and a biosensing textile as set forth in the appended claims. Other features of the invention will be apparent from the dependent claims, and the description which follows.

According to a first aspect of the present disclosure, there is provided a method of manufacturing a biosensing textile. The method comprises providing a textile patch comprising a biosensing unit for measuring a biosignal of the wearer. The method comprises providing a controller for controlling the biosensing unit on a surface of the textile patch. The method comprises attaching the textile patch to a textile panel to form the biosensing textile. The controller is sandwiched between the textile patch and the textile panel.

Here, “biosignal” may refer to any signal in a living being that can be measured and monitored. The term “biosignal” is not limited to electrical signals and can refer to other forms of non-electrical biosignals. A biosensing unit therefore refers to an electronic component that is able to measure a biosignal of the wearer. The biosensing unit may comprise one or more electrodes but is not limited to this arrangement.

Beneficially, the method enables the biosensing unit to be attached to the controller and integrated into a textile panel. In this way, the biosensing unit and the controller can be incorporated effectively and potentially seamlessly into a textile panel which may then form a garment or be incorporated into a garment.

The controller is attached to a surface of the textile panel. The attachment may not be a permanent mechanical attachment and instead the attachment may be a temporary attachment formed by providing the controller on the textile panel without forming a mechanical coupling. The textile panel may apply pressure to the controller to urge the controller towards the surface of the textile patch to form the attachment. This pressure may be applied as a result of the textile panel comprising an elastomeric material. The textile panel may comprise an elasticated pocket which houses the controller and applies the pressure to the controller. The controller can be inserted into the elasticated pocket and removed therefrom. In some examples, the textile patch and the controller may both comprise magnets or magnetic material. When provided on the surface of the textile patch, the controller may be magnetically attracted to the textile patch to thus from a releasable mechanical attachment.

The controller may be, but is not required, to be in direct contact with the surface of the textile patch. Intermediate layers such as insulating or bonding layers may separate the controller from the textile patch if desired.

Providing the controller on a surface of the textile patch may comprise forming a conductive connection between the controller and the biosensing unit. The conductive connection may be formed by a conductive material that extends through the textile patch to conductively connect the controller to the biosensing unit.

The conductive material may be part of the controller such as one or more conductive elements which extend from the controller. The conductive elements may be conductive prongs or pads for example. The conductive connection may be formed by one or more conductive projections such as studs. The one or more conductive projections may be attached to the controller and may be pushed into/through the textile patch to form the conductive connection with the biosensing unit.

The conductive connection may be formed by conductive thread which is sewn through the textile patch to conductively connect the controller to the biosensing unit. The textile patch may be conductive or may comprise conductive regions formed by, for example, conductive ink, conductive thread, or conductive paste. The textile patch may therefore form the conductive connection between the controller and the biosensing unit. That is, attaching the controller to the textile patch may form the conductive connection with the biosensing unit.

The controller may be wirelessly connected to the biosensing unit. That is, the biosensing unit may comprise a communicator for wireless communication with the controller. In this way, a conductive connection between the controller and the biosensing unit provided via/through the textile patch is not required in all examples of the present invention. The biosensing unit may comprise or be associated with a power source for powering the biosensing unit/communicator.

Providing the textile patch comprising the biosensing unit may comprise attaching the biosensing unit to a first surface of the textile patch. The biosensing unit may be welded, adhered, stitched or otherwise attached to the textile patch. Providing the controller on the textile patch may comprise providing the controller on a second surface of the textile patch opposing the first surface of the textile patch such that the biosensing unit and the controller are located on opposing sides of the textile patch. The controller may be welded, adhered, stitch or otherwise attached to the textile patch.

The biosensing unit may be integral with the textile patch. That is, the textile patch may be a textile patch biosensing unit. The biosensing unit may be formed of one or more fibres of the textile patch. That is, one or more fibres of the textile patch may be woven to form the biosensing unit. The biosensing unit may be a textile-based biosensing unit, optionally a fabric biosensing unit

The biosensing unit may be printed onto the textile patch. The biosensing unit may be formed of a conductive ink such as a silver ink. The biosensing unit may be screen printed, digitally (e.g. inkjet) printed, transfer printed, sublimation printed, pad printed, coated, transfer coated, sprayed, or extruded onto a surface of the textile. The biosensing unit may be formed of conductive inks, conductive pastes and/or conductive coatings, or any combination thereof.

Attaching the textile patch to the textile panel may comprise welding the textile patch to the textile panel. This may comprise applying heat and/or pressure to the textile patch/textile panel so as to cause the textile patch to be attached to the textile panel. Attaching the textile patch to the textile panel may comprise adhering the textile patch to the textile panel. The adhesive may be a conductive adhesive which may act to conductively connect the controller to the biosensing unit. Attaching the textile patch to the textile panel may comprise stitching the textile patch of the textile panel. The stitching may comprise conductive threads which conductively connect the controller to the biosensing unit.

The textile patch may comprise one or more recesses arranged to receive conductive components.

The textile patch may be a fabric patch. The textile panel may be a fabric panel.

The textile panel may be shaped to position the biosensing unit away from the garment such that, when worn, the biosensing unit is positioned on or near the body surface. This means that the textile panel has a three-dimensional shape. Beneficially, the textile panel is shaped to urge the biosensing unit towards the body surface when worn. This helps maintain the biosensing unit in close proximity to the body surface and, in some applications, in contact with the body surface. The textile panel may comprise a dart, wherein the dart acts to shape the textile. The textile panel may comprise a seam, wherein the seam acts to shape the textile.

The biosensing textile panel may further comprise a second textile panel; and a biosensing unit positioned on the second textile panel. The second textile panel may be joined to the other (first) textile panel. The second textile panel may be shaped to position the biosensing unit away from the garment such that, when worn, the biosensing unit is positioned on or near the body surface. The second textile panel may comprise a dart, wherein the dart acts to shape the textile panel. The second textile panel may comprise a seam, wherein the seam acts to shape the textile panel.

The textile panel may be bias cut. The first textile panel and/or the second textile panel may be bias cut.

The biosensing unit may be positioned on the textile panel at a location corresponding to a left chest region of the wearer such that, when worn, the biosensing unit is positioned proximate to the cardiac region of the wearer. The biosensing textile may further comprise a second biosensing unit positioned on the textile panel, wherein the second biosensing unit may be located at a position corresponding to a central chest region of the wearer such that, when worn, the second biosensing unit is proximate to the central chest region of the wearer.

The biosensing textile may further comprise a power source or a plurality of power sources. The power source may be provided on the textile panel. The power source may comprise an indicator arranged to indicate a status of the power source. The textile panel may comprise a holder arranged to releasably hold the power source. The power source may be incorporated into or have the appearance of a fastener. The fastener may be a button, clasp, toggle, stud, snap fastener, popper, eyelet or, buckle.

The biosensing textile may comprise a plurality of biosensing units optionally disposed on the textile panel.

The biosensing unit may comprise one or more electrodes. The biosensing unit may form a textile patch electrode such as a fabric patch electrode. The biosensing unit may comprise one or more electrodes and one or more conductive pathways extending from the electrode to one or more connection terminals. The controller may contact the connection terminals so as to receive measurement signals from the electrodes via the conductive pathway.

The biosensing unit may be used for measuring one or a combination of bioelectrical, bioimpedance, biochemical, biomechanical, bioacoustics, biooptical or biothermal signals of the wearer. The bioelectrical measurements include electrocardiograms (ECG), electrogastrograms (EGG), electroencephalograms (EEG), and electromyography (EMG). The bioimpedance measurements include plethysmography (e.g., for respiration), body composition (e.g., hydration, fat, etc.), and electroimpedance tomography (EIT). The biomagnetic measurements include magnetoneurograms (MNG), magnetoencephalography (MEG), magnetogastrogram (MGG), magnetocardiogram (MCG). The biochemical measurements include glucose/lactose measurements which may be performed using chemical analysis of the wearer's sweat. The biomechanical measurements include blood pressure. The bioacoustics measurements include phonocardiograms (PCG). The biooptical measurements include orthopantomogram (OPG). The biothermal measurements include skin temperature and core body temperature measurements. The power source may be conductively connected to the controller by a conductor.

The power source may be a battery. The battery may be a rechargeable battery. The battery may be a rechargeable battery adapted to be charged wirelessly such as by inductive charging. The power source may comprise an energy harvesting device. The energy harvesting device may be configured to generate electric power signals in response to kinetic events such as kinetic events performed by a wearer of a garment that the biosensing textile layer forms or is incorporated into. The kinetic event could include walking, running, exercising or respiration of the wearer. The energy harvesting material may comprise a piezoelectric material which generates electricity in response to mechanical deformation of the converter. The energy harvesting device may harvest energy from body heat of a wearer of a garment that the biosensing textile layer forms or is incorporated into. The energy harvesting device may be a thermoelectric energy harvesting device.

The biosensing textile may further comprise a communicator arranged to communicate with an external device over a wireless network.

The communicator may be a mobile/cellular communicator operable to communicate the data wirelessly via one or more base stations. The communicator may provide wireless communication capabilities for the garment/textile and enables the garment to communicate via one or more wireless communication protocols such as used for communication over: a wireless wide area network (WWAN), a wireless metroarea network (WMAN), a wireless local area network (WLAN), a wireless personal area network (WPAN), Bluetooth® Low Energy, Bluetooth® Mesh, Bluetooth® 5, Thread, Zigbee, IEEE 802.15.4, Ant, a near field communication (NFC), a Global Navigation Satellite System (GLASS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network. A plurality of communicators may be provided for communicating over a combination of different communication protocols.

The communicator may be incorporated into the controller. The communicator may be provided on the textile panel such as at a location different to that of the controller.

The textile panel and/or the textile patch may be constructed from a woven or a non-woven material. The textile panel and/or the textile patch may be a fabric. The textile panel and/or the textile patch may be formed from yarn. The yarn may be a natural fibre, or a natural fibre blended with one or more other materials which can be natural or synthetic or a synthetic fibre. The yarn may be cotton. The cotton may be blended with polyester and/or viscose and/or polyamide according to the particular application. Silk may also be used as the natural fibre. Cellulose, wool, hemp and jute are also natural fibres that may be used in the textile. Polyester, polycotton, nylon and viscose are synthetic fibres that may be used in the textile

The biosensing textile may form or be incorporated in a biosensing garment. The textile panel may form an outer layer of the biosensing garment.

The textile panel may comprise a mesh material or a webbing material.

According to a second aspect of the present disclosure, there is provided a method of manufacturing a biosensing garment. The method comprises manufacturing a biosensing textile using the method according to the first aspect of the disclosure. The method comprises providing a garment. The method comprises disposing the biosensing textile inside the garment. The method comprises attaching the biosensing textile to the inside of the garment.

Beneficially, the present disclosure provides a biosensing garment with an inner biosensing textile. The inner biosensing textile comprises components for performing biosensing and, in particular, comprises a biosensing unit for performing a biosensing measurement. These components for performing biosensing are therefore not visible from the outside of the garment and do not affect the outward appearance of the garment.

Attaching the biosensing textile to the inside of the garment may comprise attaching a first region of the textile panel to the garment such that the first region is unable to move relative to the garment. A second region of the textile panel may be able to move relative to the garment.

Significantly still, a first region of the biosensing textile is attached to the garment while another region of the biosensing textile is able to move relative to the garment. Beneficially, this means that a part of the textile is attached to the garment while another part is free to move relative to the garment. This means that the biosensing textile does not pull on the garment when the wearer moves as part of the textile is able to move relative to the garment. As such, the inner biosensing textile is able to perform the required biosensing measurements with a minimal effect on the comfort of the wearer even during wearer motion. In addition, as the biosensing textile does not or does not significantly pull on the garment, the biosensing textile has a minimal effect on the outward appearance of the garment.

The first portion of the textile may be an end region of the textile panel. The second portion of the textile may be a remaining portion of the textile panel. That is, the remaining portion of the textile panel may be able to move relative to the garment. This may mean that only the end portion of the textile panel is unable to move relative to the garment.

The biosensing textile may be a first biosensing textile. The method may further comprise manufacturing a second biosensing textile according to the first aspect of the disclosure; disposing the second biosensing textile inside the garment; and attaching the second biosensing textile to the inside of the garment.

The first biosensing textile may be attached to the second biosensing textile. The first textile and second textile may together form a loop or U-shaped band that is attached at both ends to a region of the garment. The region of the garment may be the shoulder region of the garment. The second textile and the first textile may together define an aperture through which a part of the wearer's body may be received. The aperture may be an arm hole for receiving an arm of the wearer. The aperture may be a leg hole for receiving a leg of the wearer.

The garment may be a free-form garment. A free-form garment will be understood as referring to a garment that is not skin-tight and is not a compression garment.

The garment may be a top. The first region of the textile panel may be attached to the top at a position corresponding to a shoulder of the wearer such that, when worn, the first region of the textile panel is proximate to the shoulder of the wearer.

The garment may be constructed from a woven or a non-woven material. The garment may be constructed from natural fibres, synthetic fibres, or a natural fibre blended with one or more other materials which can be natural or synthetic. The yarn may be cotton. The cotton may be blended with polyester and/or viscose and/or polyamide according to the particular application. Silk may also be used as the natural fibre. Cellulose, wool, hemp and jute are also natural fibres that may be used in the textile. Polyester, polycotton, nylon and viscose are synthetic fibres that may be used in the textile

The garment may be a top. The top may be a shirt, t-shirt, blouse, sweater, jacket/coat, or vest. The garment may be a dress, brassiere, shorts, pants, arm or leg sleeve, vest, jacket/coat, glove, armband, underwear, headband, hat/cap, collar, wristband, stocking, sock, or shoe, athletic clothing, swimwear, wetsuit or drysuit.

According to a third aspect of the present disclosure, there is provided a biosensing textile. The biosensing textile comprises a textile patch comprising a biosensing unit. The biosensing textile comprises a controller for controlling the biosensing unit, wherein the controller is provided on a surface of the textile patch. The biosensing textile comprises a textile panel. The textile panel is attached to the textile patch to form the biosensing textile. The controller is sandwiched between the textile patch and the textile panel.

The biosensing textile may be manufactured according to the first aspect of the present disclosure.

According to a fourth aspect of the present disclosure, there is provided a biosensing garment. The biosensing garment comprises a garment. The biosensing garment comprises a biosensing textile according to the third aspect of the present disclosure. The biosensing textile is disposed within the garment.

The biosensing garment may be manufactured according to the second aspect of the present disclosure.

The biosensing units and/or conductors in accordance with aspects of the present disclosure may be formed of a conductive metallic material such as copper, gold, or silver. The biosensing units and/or conductors may be formed of a 2D material. The 2D material may be a carbon-based material.

The carbon-based material may comprise graphene, e.g. pristine graphene, and/or reduced graphene oxide. The carbon-based material may be graphene and/or reduced graphene oxide and/or in combination with one or more additional conductive agents. The carbon-based material may be a graphene derivative. The graphene, reduced graphene oxide or graphene derivative may comprise nanoparticles, nanosheets, and microparticles.

The biosensing units and/or conductors may be printed onto a textile or coated onto a fibre/yarn of the textile. The conductive material may be incorporated by a dyeing process in which a liquid composition containing the conductive material is contacted with the fibre/yarn. Therefore, a conductive material may be incorporated into a yarn in order to produce a yarn which is capable of conducting electricity. In this way, a conductor may be formed from a fibre or yarn of the textile. This may mean that an electrically conductive materials such as silver or graphene is incorporated into the fibre/yarn. In some examples, the yarn may be a graphene yarn. That is, a yarn constructed entirely, essentially or substantially of graphene, e.g. graphene fibres.

The graphene material may comprise single layers of graphene or thin stacks of two to ten graphene layers. The thin stacks of graphene are distinguished from graphite by their thinness and a difference in physical properties. In this regard, it is generally acknowledged that crystals of graphene which have more than 10 molecular layers (i.e. 10 atomic layers which equates to a thickness of approximately 3.5 nm) generally exhibit properties more similar to graphite than to graphene. Thus, throughout this specification, the term graphene may mean a carbon nanostructure with up to ten graphene layers. Similarly, the reduced graphene oxide may be present as single layers of reduced graphene oxide or thin stacks of two to ten reduced graphene oxide layers. The conductor may be formed from flakes of graphene or reduced graphene oxide that comprise 1 to 10 layers. Each layer of graphene or reduced graphene oxide present within a flake has a length and a width dimension to define the size of the plane of the layer. Typically, the length and width of the layers are within the range of 10 nm to 2 microns. The flakes may be deposited by printing an electrically conductive ink formulation that comprises flakes of graphene or graphene oxide. The printing may be performed using screen printing or digital (e.g. inkjet) printing. Digital printing, and in particular inkjet printing provides a simple and efficient way of producing the electrically conductive materials.

In some examples. there may be additional electrically conductive agents present in one or more of the conductors, such as metallic components (e.g. silver precursor, silver nanoparticles, carbon nanotubes, or poly(3,4-ethylenedioxythiophene) polystyrene sulfonate (PEDOT/PSS)).

The conductors may be formed of conductive transfers. The conductive transfer may comprise a first non-conductive ink layer and a second non-conductive ink layer. An electrically conductive layer may be positioned between the first non-conductive ink layer and the second non-conductive ink layer. The conductive transfer may be adhered to the textile via use of an adhesive layer so as to form the conductor on the textile. An example conductive transfer is described in UK Patent Application Publication No. GB 2555592 (A) the disclosures of which are hereby incorporated by reference. The electrically conductive layer may comprise graphene.

### BRIEF DESCRIPTION OF THE DRAWINGS

Examples of the present disclosure will now be described with reference to the accompanying drawings, in which:

FIGS. 1A-1E shows a series of views representing stages by which an example biosensing textile is formed and attached to a garment;

FIG. 2 shows a side view of biosensing textile attached to a garment as shown in FIG. 1E;

FIG. 3 shows a front view of an example textile patch according to aspects of the present disclosure;

FIGS. 4A-4D shows a series of views representing stages by which an example biosensing textile is formed and attached to a garment;

FIG. 5 shows a side view of biosensing textile attached to a garment as shown in FIG. 4D; and

FIG. 6 shows a sectional view of an example biosensing garment according to aspects of the present invention.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

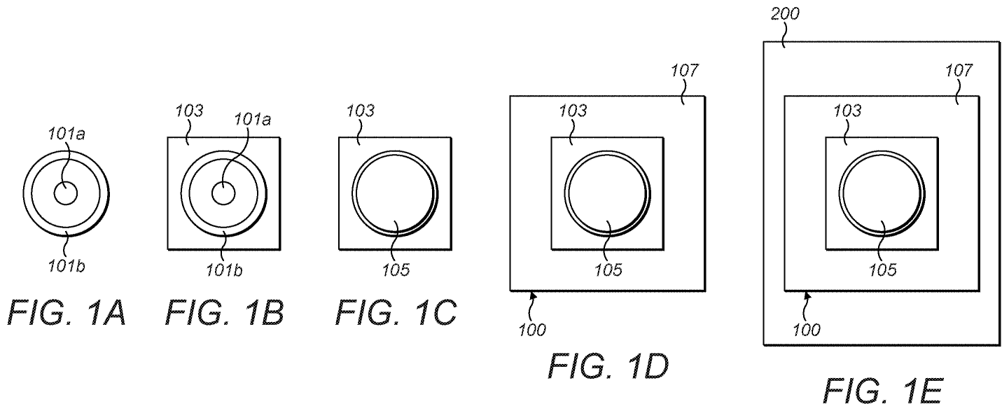

Referring to FIGS. 1A to 1E, there is shown a series of views showing stages by which an example biosensing textile 100 is formed and attached to a garment 200 according to aspects of the present disclosure.

Referring to FIG. 1A, there is shown a biosensing unit in the form of an electrode 101a, 101b. The following examples all refer to biosensing units in the form of electrodes, but the present invention is not limited to this arrangement and other forms of biosensing unit may be used in the aspects of the present disclosure. The electrode 101a, 101b comprises a first electrical contact 101a and a second electrical contact 101b. The first and second electrical contacts 101a, 101b are arranged as concentering rings.

Referring to FIG. 1B, there is shown a textile patch 103 on which the electrode 101a, 101b is provided. The electrode 101a, 101b is provided on a first surface of the textile patch 103.

Referring to FIG. 1C, there is shown a controller 105 for controlling the electrode 101a, 101b. The controller 105 is provided on the textile patch 103 and, in particular, is provided on a second surface of the textile patch 103 that is opposite to the first surface of the textile patch 103 on which the electrode 101a, 101b is provided. Conducting studs extend through the textile patch 103 to connect the controller 105 to the electrode 101a, 101b. In this way, the controller 105 is conductively connected to the electrode 101a, 101b for controlling the electrode 101a, 101b and the controller 105 and the electrode 101a, 101b are attached to the textile patch 103.

Referring to FIG. 1D, there is shown a textile panel 107 on which the textile patch 103 is provided to thus form the biosensing textile 100. The textile patch 103 is welded onto the textile panel 107. Welding the textile patch 103 onto the textile panel 107 involves applying heat and pressure to join the textile patch 103 to the textile panel 107. In other examples, adhesive may be used to join the textile patch 103 to the textile panel 107 or the textile patch 103 may be stitched or otherwise joined to the textile panel 107.

Referring to FIG. 1E, there is shown the biosensing textile 100 attached to a garment 200. The biosensing textile 100 is attached to the inside surface of the garment 200.

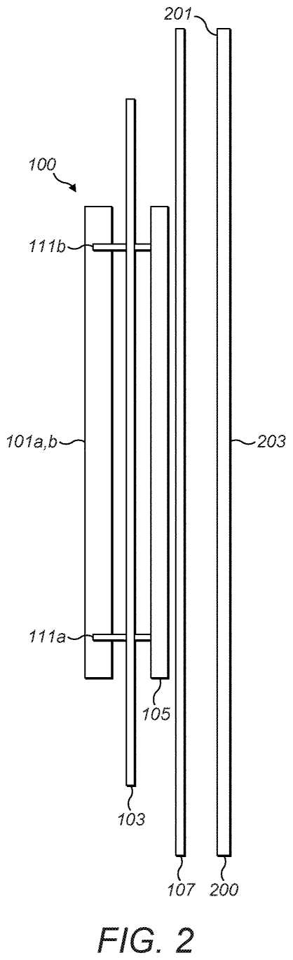

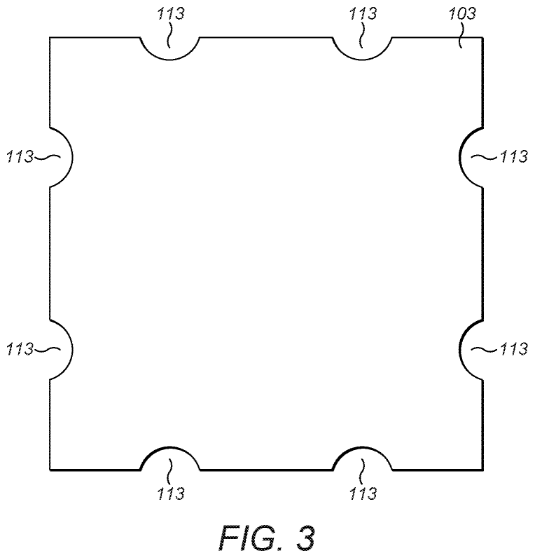

Referring to FIG. 2, there is shown a side view of the garment 200 and biosensing textile 100 as shown in FIG. 1E. In FIG. 2, it can be seen that the controller 105 and the electrode 101a,b are positioned on opposite sides of the textile patch 103 and are connected together via conductive studs 111a, 111b that project through the textile patch 103. The textile patch 103 is welded onto the textile panel 107 and the textile panel 107 is attached to the garment 200. In this way, the biosensing textile 100 is attached to the inside surface 201 of the garment 200 opposite to the outside surface 203 of the garment 200. In this way, the biosensing textile 100 is disposed inside the garment 200. The electrode 101a, 101b is positioned on the inner surface of the biosensing textile 100 such that the electrode 101a, 101b is furthest away from the garment 200. This means that, when worn, the electrode 101a, 101b is proximate to and may be in contact with the body surface.

Referring to FIG. 3, there is shown another example of a textile patch 103. In this example, the textile patch 103 comprises recesses 113 through which electronic components such as conductors may pass through to connect the controller 105 (FIG. 2) to the electrode 101a, 101b (FIG. 2).

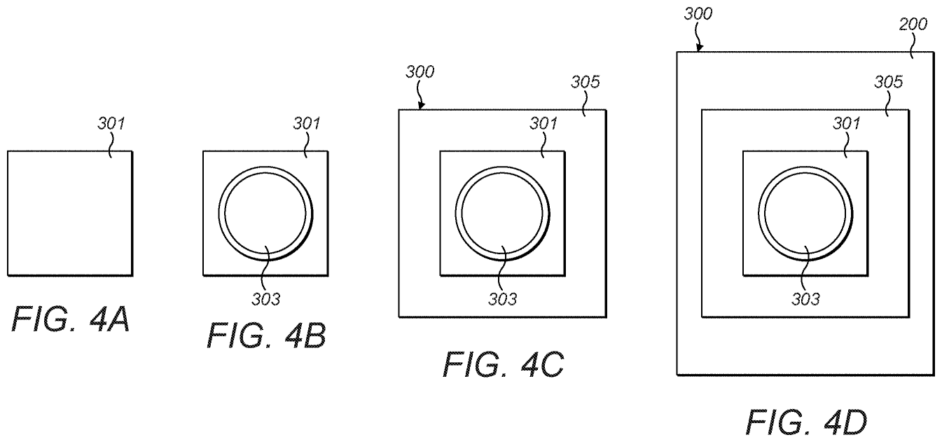

Referring to FIGS. 4A to 4D, there is shown a series of views showing stages by which an example biosensing textile 300 is formed and attached to a garment 200 according to aspects of the present disclosure.

Referring to FIG. 4A, there is shown an electrode 301. The electrode 301 is formed of a conductive textile patch 301. That is, the electrode 301 is an electrode textile patch 301. The electrode textile patch 301 may have recesses similar to the recesses shown in the textile patch of FIG. 3 to allow conductive components to pass through the electrode textile patch 301.

Referring to FIG. 4B, there is shown a controller 303 for controlling the electrode 301 The controller 303 is provided on a surface of the electrode textile patch 301. Conductive pins or other conductive elements are used to attach the controller 303 to the electrode textile patch 301 and maintain the controller 303 in conductive communication with the electrode textile patch 301. In other examples, the controller 303 may be adhesively attached to the electrode textile patch 301. In other examples, the controller 303 may be stitched to the electrode textile patch 301 with conductive thread so as to join and electrically connect the controller 303 to the electrode textile patch 301. A circuit board of the controller 303 may have apertures through which the conductive thread may pass to join the controller 303 to the electrode textile patch 301.

Referring to FIG. 4C, there is shown a textile panel 305 on which the electrode textile patch 301 is provided to thus form the biosensing textile 300. The electrode textile patch 301 is welded onto the textile panel 305. In other examples, adhesive may be used to join the textile patch 301 to the textile panel 305 or the textile patch 301 may be stitched or otherwise joined to the textile panel 305.

Referring to FIG. 4D, there is shown the biosensing textile 300 attached to a garment 200. The biosensing textile 300 is attached to the inside surface of the garment 200.

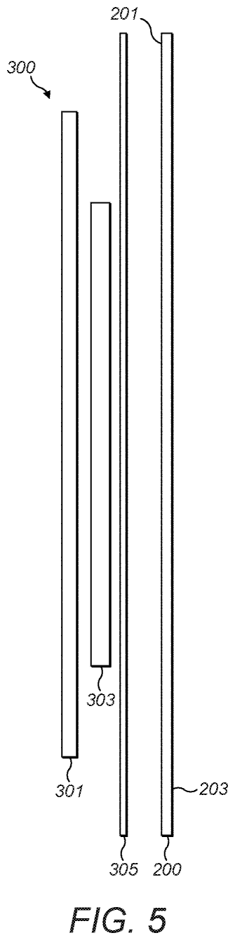

Referring to FIG. 5, there is shown a side view of the garment 200 and biosensing textile 300 as shown in FIG. 4D. In FIG. 5, it can be seen that the controller 303 is sandwiched between the electrode textile patch 301 and the textile panel 305. The textile patch 301 is welded onto the textile panel 305 and the textile panel 305 is attached to the garment 200. In this way, the biosensing textile 300 is attached to the inside surface 201 of the garment 200 opposite to the outside surface 203 of the garment 200. In this way, the biosensing textile 300 is disposed inside the garment 200. The electrode textile patch 301 is positioned on the inner surface of the biosensing textile 300 such that the electrode 301 is furthest away from the garment 200. This means that, when worn, the electrode 301 is proximate to and may be in contact with the body surface.

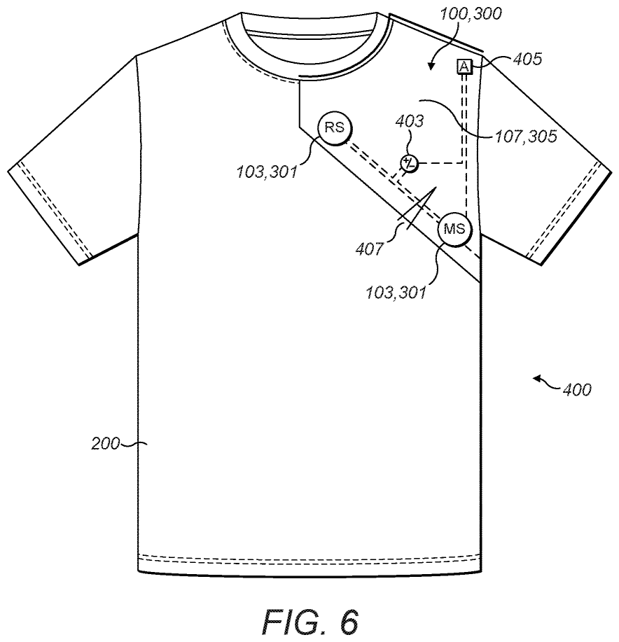

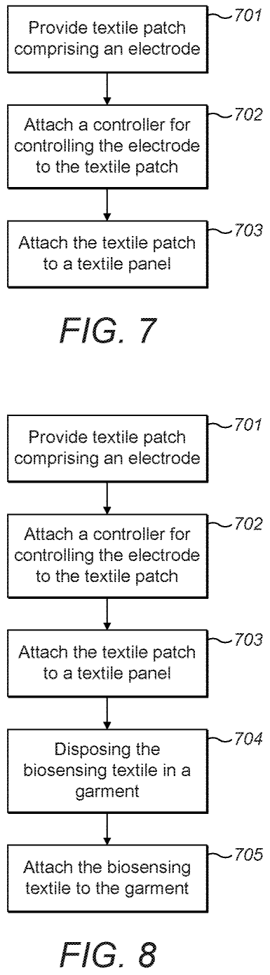

Referring to FIG. 6, there is shown a simplified sectional view of a biosensing garment 400 according to aspects of the present disclosure. The biosensing garment 400 comprises a garment 200 in the form of a T-shirt 200. The T-shirt 200 comprises a main body, a left sleeve, a right sleeve, and a collar. The T-shirt 200 is a free-form garment. By this it is meant that the T-shirt 200 is loose, not skin-tight, and not a compression garment.

The biosensing garment 400 comprises biosensing textile 100, 300 (FIG. 2, FIG. 5) disposed within the garment 200. The biosensing textile 100, 300 is not visible from the outside of the garment 200 and thus does not or does not significantly affect the external appearance of the garment 200.

The biosensing textile 100, 300 comprises a textile panel 107, 305. A first end region of the panel 107, 305 is attached to the garment 200 while the remaining portions of the panel 107, 305 are not attached to the garment 200. This means that while the first end region of the panel 107, 305 is not able to move relative to the garment 200, the remaining regions of the panel 107, 305 are able to move relative to the garment 200. The biosensing textile 100, 300 is therefore able to move freely relative to the garment 200. The panel 107, 305 does not pull on the garment 200 when the wearer moves. This means that the panel 107, 305 does limit the wearer's mobility and does not affect the outward appearance of the garment 200.

In the example of FIG. 6, the first end region is the top end region of the panel 107, 305 that is attached to the shoulder region and part of the collar region of the garment 200. The remaining portions of the panel 107, 305 are not connected to the garment 200. In this example, the bottom end region and the side regions are free ends, i.e. they are not attached to the garment 200. Beneficially, this means that the panel 107, 305 is attached to the garment 200 at positions corresponding to the shoulder region of the wearer. The shoulder region of the wearer is generally subject to little to no motion even during strenuous exercise. As such, the attachment of the panel 107, 305 to the garment 200 causes little or no pull on the garment 101 even during motion of the wearer.

The panel 107, 305 comprises a plurality of textile patches 103, 301 comprising electrodes such as the textile patches 103, 301 comprising electrodes 101a,b, 301 as described in the examples of FIG. 2 and FIG. 5. A first of the textile patches 103, 301 is located at a central upper chest region of the panel 107, 305. The “central upper chest region” will be understood as referring to a region which, when worn, corresponds to a central upper chest region of the wearer. Beneficially, when provided in this position, the weight of the textile patch 103, 301 causes the panel 107, 305 to hang downwards and urge the electrode of the textile patch 103, 301 towards the body surface. In this way, the attachment of the panel 107, 305 to the garment 200 causes the electrode to be positioned towards or near the body surface so that the electrode may measure biosignals of the wearer.

A second of the textile patches 103, 301 is located at a lower left chest region of the panel 107, 305. The “lower left chest region” will be understood as referring to a region which, when worn, corresponds to a lower left chest region of the wearer which is proximate to a cardiac region of the wearer. The panel 107, 305 is shaped to position the electrode of the textile patch 103, 301 away from the garment 200. In this way, when worn, the electrode is positioned on or near the body surface. The shaping of the panel 107, 305 is achieved through use of a dart 407 in the panel 107, 305. The dart 407 will be understood as referring to a fold that is sewn or otherwise introduced into the panel 107, 305 to provide the shape to the panel 107, 305. The panel 107, 305 may be thought of as having a flat, planar, surface. The dart 407 has the effect of removing a wedge-shaped piece of the panel 107, 305 and pulling the edges of that wedge together to create a shallow cone. In this way, the dart 407 urges the electrode away from the main planar surface of the panel 201.

The dart 407 is not required in all examples of the present disclosure, and instead other structures or features of the panel 107, 305 may be used to provide the desired shape to the panel 107, 305 to position the electrode away from the garment 200. For example, a seam, pleat, or gather in the panel 107, 305 may be used to provide the same effect as the dart 407.

The panel 107, 305 may be bias cut. This means that that a piece of textile forming the panel 107, 305 is cut diagonally or obliquely to the grain of the textile. Being cut on the bias means that the panel 107, 305 has more stretch when compared to textiles cut along the straight grain or cross grain. Being bias cut means that the panel 107, 305 will drape in a way which contours to the shape of the body surface. This helps maintain the electrodes in a position which is near or in contact with the body surface.

A first electrode may act as a reference electrode. The controller in communication with the first electrode may act as a reference controller. A second may act as a measuring electrode. The controller in communication with the second electrode may act as a measuring controller. That is, one of the first and second electrodes may be controlled to act as a reference during biopotential and/or bioimpedance measurements.

The first controller and the second controller are able to stimulate the body, such as by injecting a current into the body via the electrode(s) for performing an impedance measurement. The first controller and the second controller are also able to measure a physiological signal of the body, such as an ECG, by measuring a potential via the electrode(s). The first electrode and the second electrode may both comprise a first electrical contact and a second electrical contact which are spaced apparat from another. The first and second electrical contacts may be arranged as concentric rings, for example. The potential may be measured between the electrical contacts of the first electrode and/or the second electrode.

The biosensing textile 100, 300 further comprises a communicator 405. The communicator 405 transmits biometric data recorded by the electrodes and optionally processed by the first/second controller wirelessly to an external device. In some examples of the present disclosure, the communicator 405 is a cellular communicator 405 operable to communicate the biometric data wirelessly with an external server via one or more base stations

The communicator 405 is conductively connected to the second controller by a conductor. The first electrode and/or first controller are conductively connected to the second electrode and/or second controller via a conductor.

The communicator 405 in this example is shown at a position which is spaced apart from the first and second electrodes at a position close to the end region of the textile panel 107, 305. In some examples, the communicator 405 may be incorporated with one of the controllers or the electrodes.

The textile panel 107, 305 further comprises a power source 403 for powering the first controller and the second controller. The power source 403 may be a battery 403. The power source 403 is conductively connected to the first controller by a conductor. The power source 403 is conductively connected to the second controller by a conductor. The power source 403 is conductively connected to the communicator 405 by a conductor. In other examples, a separate power source is provided for each of the controllers. That is, a first power source may be provided for powering the first controller and a second power source may be provided for powering the second controller.

The conductors are, in this example, formed of a graphene or a graphene-derivative and are printed onto the textile 107, 305 using a screen-printing process. Other printing processes may be used. In some examples, the conductor may be a conductive transfer. The conductive transfer may comprise graphene.

It will be appreciated that the present disclosure is not limited to screen printing conductors onto a textile or the use of conductive transfers. In other examples, the conductors may be incorporated into one or more fibres of the textile.

The first electrode and second electrode may be conventional metallic electrodes such as silver/silver chloride (Ag/AgCl) electrodes.

The first and second electrodes in may be formed of a 2D electrically conductive material. The material may be graphene or graphene-derivative which is screen printed onto the textile. The combination of the electrodes being integrated into the textile and formed of a 2D electrically conductive material means that the electrodes have a minimal footprint on the textile.

The garment may comprise an aperture through which the power source of the inner biosensing layer is visible. The aperture may be sized to receive the power source such that the power source is accessible via the outside surface of the garment. The power source may be removable from the textile panel. The textile panel may comprise a holder for receiving the power source. The power source may snap in/out of the holder or may clip in/out of the holder. The power source may visually indicate the status of the power source such as by indicating the amount of charge remaining for the power source. The power source may comprise one or more light sources for indicating the status of the power source.

Referring to FIG. 7, there is shown an example method of manufacturing a biosensing textile according to aspects of the present disclosure. Step 701 of the method comprises providing a textile patch comprising an electrode. Step 702 of the method comprises providing a controller for controlling the electrode on a surface of the textile patch. Step 703 of the method comprises attaching the textile patch to a textile panel to form the biosensing textile such that the controller is sandwiched between the textile patch and the textile panel. Step 703 may be performed before step 702. That is, the controller may be provided on a surface of the textile patch after the textile patch is attached to the textile panel.

Referring to FIG. 8, there is shown an example method of manufacturing a biosensing garment according to aspects of the present disclosure. Step 701 of the method comprises providing a textile patch comprising an electrode. Step 702 of the method comprises providing a controller for controlling the electrode on a surface of the textile patch. Step 703 of the method comprises attaching the textile patch to a textile panel to form the biosensing textile such that the controller is sandwiched between the textile patch and the textile panel. Step 704 of the method comprises disposing the biosensing textile inside a garment. Step 705 of the method comprises attaching the biosensing textile to the inside of the garment.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of manufacturing a biosensing textile, the method comprising:
providing a textile patch comprising a biosensing unit;
attaching the textile patch to a textile panel to form the biosensing textile; and
providing a controller for controlling the biosensing unit on a surface of the textile patch,
wherein the controller is sandwiched between the textile patch and the textile panel.

2. The method as claimed in claim 1, wherein providing the controller on a surface of the textile patch comprises forming a conductive connection between the controller and an electrode.

3. The method as claimed in claim 1, wherein providing the textile patch comprising the biosensing unit comprises attaching the biosensing unit to a first surface of the textile patch, and wherein providing the controller comprises providing the controller on a second surface of the textile patch opposing the first surface of the textile patch such that the biosensing unit and the controller are located on opposing sides of the textile patch.

4. The method as claimed in claim 1, wherein the biosensing unit is integral with the textile patch.

5. The method as claimed in claim 4, wherein the biosensing unit is formed of one or more fibres of the textile patch.

6. The method as claimed in claim 1, wherein attaching the textile patch to the textile panel comprises welding the textile patch to the textile panel.

7. The method as claimed in claim 1, wherein attaching the textile patch to the textile panel comprises adhering the textile patch to the textile panel.

8. The method as claimed in claim 1, wherein attaching the textile patch to the textile panel comprises stitching the textile patch of the textile panel.

9. The method as claimed in claim 1, wherein the textile patch comprises one or more recesses arranged to receive conductive components.

10. The method as claimed in claim 1, wherein the textile patch is a fabric patch, and optionally wherein the textile panel is a fabric panel.

11. A biosensing textile, comprising:
a textile patch comprising a biosensing unit;
a controller for controlling the biosensing unit, wherein the controller is provided on a surface of the textile patch;
a textile panel, wherein the textile panel is attached to the textile patch to form the biosensing textile, and
wherein the controller is sandwiched between the textile patch and the textile panel.

12. The biosensing textile as claimed in claim 11, wherein the controller is electrically connected to the biosensing unit by a conductive material that extends through the textile patch.

13. The biosensing textile as claimed in claim 12, wherein the biosensing unit is attached to a first surface of the textile patch, and wherein the controller is provided on a second surface of the textile patch opposing the first surface of the textile patch such that the biosensing unit and the controller are located on opposing sides of the textile patch.

14. The biosensing textile as claimed in claim 12, wherein the biosensing unit is adhered to the textile patch.

15. The biosensing textile as claimed in claim 12, wherein the textile patch comprises one or more recesses arranged to receive conductive components.

16. The method as claimed in claim 2, wherein providing the textile patch comprising the biosensing unit comprises attaching the biosensing unit to a first surface of the textile patch, and wherein providing the controller comprises providing the controller on a second surface of the textile patch opposing the first surface of the textile patch such that the biosensing unit and the controller are located on opposing sides of the textile patch.

17. The biosensing textile as claimed in claim 13, wherein the biosensing unit is attached to a first surface of the textile patch, and wherein the controller is provided on a second surface of the textile patch opposing the first surface of the textile patch such that the biosensing unit and the controller are located on opposing sides of the textile patch.
